(19) United States
(12) Patent Application Publication
(10) Pub. No.: US 2004/0202640 A1
(43) Pub. Date: Oct. 14, 2004

(54) Method for topical treatment of scars with rotein Kinase C inhibitors

(76) Inventor: Wilson Trafton Crandall, Fort Defiance, VA (US)

Correspondence Address: WILSON T. CRANDALL, P O BOX 346, VERONA, VA, 24482, US

(21) Appl. No.: 10/410,667

(22) Filed: Apr. 9, 2003

(51) Int. Cl. A61K 31/685; A61K 31/74

(52) U.S. Cl. 424/78.26; 514/78

(57) ABSTRACT

This invention relates to the topical treatment of keloids, hypertrophic scars and burn scars by the use of a selected protein kinase C inhibitior, an effective penetrating agent selected from the lecithin organogel, or poloxamer 407 lecithin organogel and a polyacrylate polymer, carbomer, to eliminate the stickiness of the poloxamer 407. This encourages better compliance and allows use three times daily. The protein Kinase C inhibitors may be selected from phytosphingosine, phytospinogsine-1-phosphate, sphinganine, sphinganine-1, lysosphingoids, sphingosine, sphingosine-1-phosphate, curcuminoids, tetrahydrocurcumin, apigenin and W-7

### PRIOR RELATED APPLICATIONS

This present application is related to Provisional application 60/114,813 and U.S. Pat. No. 6,306,383

### TECHNICAL FIELD OF THE INVENTION

The present invention is related to a process and composition for topically inhibiting Protein Kinase C. More particularly, the present invention relates to topically applying the composition disclosed herein in order to treat the affected skin or underlying structures of humans and animals. Protein Kinase C inhibitors have been shown effective in vitro and in a limited fashion orally, but not efficacious when used topically except in psoriasis where the stratum corneum is abnormal. What is needed is a topical composition which is safe, cost effective and aesthetically acceptable to the patient so that compliance will be followed.

### BACKGROUND OF THE INVENTION

Mukhtar in Pharmacology of the Skin describes the communication between cells as being mediated by different biomolecules, such as hormones. These so called primary messengers bind to specific receptors on the cell surface. The binding of a primary messenger to its receptors conveys a certain information to the cell which is subsequently transduced through the membranes by a chain of signaling. This process involves various membrane structures and leads to the activation of an enzyme located at the intracellular side of the membrane. The stimulated enzyme generates a second messenger which evokes the cellular response; in most cases, by the activation of other enzymes. By these steps, the initial extracellular signal is converted into an intracellular signal. This process is called signal transduction.

The inositide cascade represents one of the several signals transducing pathways. In its course, two second messengers, diacylglycerol (DG) and inositol triposphate (IP 3), are released. DG remains in the membrane and activates protein kinase C (PKC). IP 3 acts by releasing calcium ions from their intracellular stores. The calcium ions subsequently evoke the cellular response, mainly by activating a protein kinase C (PKC) Protein kinases regulate responses by phosphorylation of substrate proteins (eg receptors or enzymes) and thereby alter their state of activity. In the case of the inositide cascade, PKC mainly performs this reaction. Sphingosine dose-dependently inhibits PKC, but also binds to calmodulin (CaM) function and therefore inhibits CaM function. Keratinocyte intercellular adhesion molecule, 1 (ICAM-1) is thought to be involved in dermal lymphocyte infiltration. The PKC activating phorbol ester, PMA has been reported to induce the expression of ICAM-1 in normal human keratinocytes. This effect can be blocked by a PKC inhibitor and suggests that PKC might play a regulatory role in ICAM-1 as well as LFA-1 expression. PKC is known to function in cutaneous tumor promotion and in that it represents the major cellular receptor for phorbol esters. PKC inhibitors like sphingosine inhibit phorbol ester induced omithine decarboxylase activity in the mouse skin.

Crandall U.S. Pat. No. 6,306,383 discloses that several PKC inhibitors treated various forms of hypertrophic scar and keloids with a transdermal vehicle, PLURONIC lecithin organogel. Whereas, women with stretch marks were pleased with the response, they complained about the stickiness of the Pluronic 127. In that it was used over the entire abdomen it also soiled their clothes. Open heart surgery patients with hypertrophic sternotomy scars had the same complaint, as did breast reduction subjects. Protein kinase C is very responsive to concentration of the inhibitor. Poor compliance will definitely compromise the response to the Rx. Clearly an improvement of the formulation had to be made so that an aesthetic quality was present so that compliance would be followed and the best clinical response could be obtained.

What is needed is a method and composition which is efficacious in the treatment of scar, but would eliminate the stickiness of the PLURONIC. This improvement in the aesthetics of the method and composition is an improvement over U.S. Pat. No. 6,306,383.

### SUMMARY OF THE INVENTION

The present invention provides a composition and easy to use therapeutic method for inhibiting PKC activity in the skin, muco-cutaneous junction and underlying structures, as well as disease processes related to it. These processes include, but are not limited to, cutaneous tumor, rheumatoid arthritis, cell mediated hyper immunity, hypertrophic scar, keloid, carpal tunnel disease 2^{nd} to fibrosis, vitiligo, papilloma virus warts, and any inflammatory conditions in the skin or underlying structures which involve PKC.

The present invention includes a composition for topical treatment of the effects of PKC activity as a second messenger wherein the molecule or molecules are selected from the group consisting of phytosphingosine, phytosphingosine-1-phosphate, phytosphingosine hydrochloride and their metabolites, derivatives and analogues, lysosphingoids, and their metabolites, derivatives, and analogues, sphingosine, sphingosine-1 phosphate, and their metabolites, derivatives and analogues, sphinganine, sphinganine-1, and their metabolites, derivatives and analogues, curcuminoids, and their metabolites, analogs and derivatives, tetrahydrocurcumin and its analogues and derivatives, apigenin and its metabolites, derivatives and analogues or W-7 and its metabolites, derivatives and analogues. combined with delivery vehicles and penetrating agents optionally containing lecithin, unhdyrogenated or hydrogenated, lecithin organogel, Pluronic 127 lecithin organogel and an effective polyacrylate polymer. All of the actives are considered adequate to practice the invention.

Crandall U.S. Pat. No. 6,306,383 mentions gelling agents and specifically polyacrylates. Carbomer is used as a thickening agent at very low concentrations (less than 1%) to produce a wide range of viscosities and flow properties in topical lotions, creams and gels and in transdermal gel reservoirs. The use of the carbomer was not referred to in the capacity it is used in the current invention and is an improvement when 1 or more of the polyacrylate gels, (NOVEON, Cleveland, Ohio) gels are used with the poloxamer 407 lecithin organogel to eliminate the stickiness of the poloxamer 407 and thereby increase compliance and prevent excoriation by children.

Therapeutically effective amounts of phytosphingosine may be combined at concentrations of 0.01 g to 7.5 g per 100 grams of PLO formulation for topical application of the composition of this invention.

Sphingosine had not been available commercially unless it was prepared synthetically and this was not cost effective. Shortly a safe form of sphingosine and sphingosine 1-phosphate will be available by yeast fermentation. It is believed, Jefferson & Schulman Dept. Pharmacology, Stanford School of Medicine, 1988. Sphingosine was thought to block the effects of both the Ca +2 calmodulin complex and of diacygylcerol, as such may be a very effective inhibitor of both branches of t the phosphatidyinositol signaling pathway.

Therapeutically effective amounts of sphingosine may be used at concentrations of 0.01 g to 5 g of PLO formulation for topical application of the composition of this invention. sphingosingosine-1-phosphate under varying conditions can promote apoptosis and promote tumor. However, it is interesting that it is a more effective PKC inhibitor than sphingosine and is the metabolite of sphingosine. Concentrations of 0.01 to 5 g per 100 g may be used in the formulation for topical application of this invention. However, the possibility of tumor promotion precludes the use of spingosine-1-phosphate

Therapeutically effective amounts of sphinganine and sphinganine-1 may be used at concentrations of 0.01 to 7.5 g per 100 grams of PLO formulation for topical application of the composition of this invention.

Therapeutically effective amounts of tetrahydrocurcumin may be used at concentrations of 0.01 g to 10.0 g per 100 g of PLO formulation for topical application of the composition of this invention.

The dried rhizome of Curcuma longa is a rich source of phenolic compounds or “curcuminoids”. Curcuminoids refers to a group of phenolics present in tumeric, which are chemically related to its principal ingredient, curcumin, demethoxycurcumin, and bisdemethoxycurcumin. All three impart the yellow pigmentation of the Curcuma longa plant which would be objectionable on the skin. Curcumin is first biotransformed to dihydrocucrcumin and tetrahydrocucrumin and tetrahdrocurcumin-glucuronide and are the major metabolites of curcumin.

Tetrahydrocurcumin is also obtained as a hydrogenated product of curcumin produced by reducing curcumin in an organic solvent using a metal catalyst. 0.1 g to 10 g of tetrahydrocurcumin per 100 g of PLO formulation is an effective PKC inhibitor and has the advantage of inhibiting tumor necrosis factor alpha 2^{nd} to the inhibition of the lipoxyoxygenase and cyclooxygenase pathways.

Sodium curcumin is a synthetic salt. Recently, Hahm ER et. al. described he synthesis of 12 symmetrical curcuminoids. BJC005, CHCO11, and CHCOO7. BJCOOR has shown a more powerful profile than momordin as an inhibitor of Fos-Jun inhibitor. High inhibitory activity against transcription activity was evident in the novel BJCO05 and CHC007. All of the actives are considered adequate to practice the invention

### DETAILED DESCRIPTION OF THE INVENTION

In this invention these phenolics, curcumin Bis(4_-hydroxy-3_-Methoxycinnamoyl) methane; 1,7-Bis(4_-hydroxy-3_-Methoxyphenyl)-Hepta-1,6-diene-3,5-dione); its biological and chemical reaction products, including metabolites, degradation, augmentation, and functionalized derivatives, pathways and pro drugs capable of transformation by the body into compounds having the aforementioned activity and effect analogs, and pro drugs capable of transformation by the body into compounds having the aforementioned activity and effect. Whether ketones. esters, acids, or alcohols; saturated, unsaturated, or polyunsaturated; conjugated, homoconjugated, or unconjugated; phenolic or not, capable of mimicking the action of curcumin inhibiting the up regulation of Protein Kinase C, inhibiting the activation of T & B lymphocytes, inhibiting LFA-1 and ICAM-1 inhibiting the cyclooxygenase & lipoxygenase pathways from the production of prostaglandins and tumor necrosis factor alpha is inhibited as a result of the inhibition of the lipoxyoxygenase and cyclooxygenase

The present invention provides a composition comprising a pharmaceutically effective penetrating agent, and a method for transdermally administering Protein Kinase inhibitors topically The composition and method of the present invention may be used to treat hypertrophic scar, keloid, rheumatoid arthritis, papilloma virus warts, cutaneous tumor, and any inflammatory or proliferative disease of the skin or structures underneath the skin.

The ceramides which are present in the compositions of the invention are understood to have a structure which icomparable to that of the ceramides identified as 1, 2, 3, 4, and 61 and 611. More specifically, the ceramides which are present in the compositions of the invention are understood to comprise ceramides in which the sphingoid base backbone is selected from the group consisting of sphingosine, phytosphingosphingosine, and sphinganine, wherein said sphingoid base backbone is acylated with an acyl or an acylloxyacyl group, wherein said acyl or acyloxylacyl group can have a variable chain length, optionally can have (additional) double bonds, optionally contain a hydroxy group and optionally can be branched.

Phytosphingosine is a preferred ceramide for this invention. However, other ceramides selected from the group N-tetracosanoyl phytosphingosine, N-stearoyl phyto- sphingosine, N-oleoyl phytosphingosine, N-linoleoyl-phytosphingosine. N-(2-hydroxytetracosanoyl) phytosphingosine, N-(2- hydroxyoctdecanoyl) phytosphingosine, N-(2 hydroxyoctadecanoyl) phytosphingosine, N-(27-stearoyloxyhepatoconsyl) phytosphingosine, N-(27-oleoyloxheptacosanoyl) phytosphingosine, N-(27-linoleoyloxyhepataconsanoyl) phytosphingosine, N-(23-stearoyloxytricosanoyl) phytosphingosine In addition sphinganine-1 and its metabolites and analogues., all of which are considered adequate to practice the invention.

Sphingoid bases (phytosphingosine, sphingosine and sphinganine) are present in the stratum corneum in their free form and as constituents of ceramides. In this invention, these lipids, their analogues, derivatives and reaction products, however, prepared: whether, prepared by chemical, or biochemical reaction, or by microbial fermentation, or isolated from natural sources: have effective performance in their use by the inhibition of protein kinase C, in particular the inhibition of LFA-1 & ICAM-1 and TGF beta, VEGF, PDGF and other growth factors involved in scar formation. As known by those highly skilled in the art and prodrugs capable of transformation by the body into compounds having the aforementioned activity and effect. In particular, ionic salts of the fore mentioned lipids are contemplated for use in this invention.

Curcuminoids refers to a group of phenolics present in tumeric, which are chemical related to its principal, curcumin. Curcumin analogs refer to sodium curcumin, tetrahydrocurcumin and triethylcurcumin. Metabolites of curcumin include hexahydrocurcumin, hexahydrocuminol, curcumin gluccuronide, curcumin sulfate. Curcumin and all the curcuminoids, the metabolites and derivatives of curcumin, are contemplated for use in this invention.

A preferred phospholipid for use in the present invention is phosphatidylcholine, unhydrogenated or hydrogenated, also known as lecithin. Stedman's medical dictionary (21sted.pg 879) defines lecithin as any group of phospholipids which upon hydrolysis yield 2 fatty acids molecules and a molecule each of glycerophosphoric acid and choline. There are several varieties of lecithin, soybean lecithin is the preferred and is the most economical. Lecithins are also found in nervous tissue, hepatic tissue, cardiac tissue and egg yolks. It is therefore understood that any reference to lecithin or phosphatidylcholine is intended to include is intended to include lecithin-like phospho-lipid compounds as is well known in the art. Examples of other phospholipids which can be used in accordance with the present invention include phosphatidylethanolamine, phosphatidylserine, phoshatidylinositol, and phosphatidic acid. A mixture of any of the above phospholipids maybe used in the present invention and are present in natural soy lecithins

A preferred penetrating agent and delivery vehicle is lecithin organogel which is a combination of lecithin, organic solvent, such as isopropyl palmitate or isopropyl myristate. Lecithin organogels have been described as vehicles that are useful in facilitating the delivery of lower molecular weight compounds transdermally (Willimann et al., Journal of Pharmaceutical Sciences 81:871-874 (1992) which is (incorporated herein by reference) The lecithin organogels are obtained by adding small amounts of water to a solution of lecithin in an organic solvent. Generally, lecithin organogels are prepared at room temperature by first dissolving lecithin in an organic solvent such as ethanol, isopropyl palmitate or isopropyl myristate and then adding enough water while stirring to obtain the desired gel.

Preparation of a variety of lecithin organogels, all of which are appropriate in practicing the invention, are described in Scartazzini, et al Journal of Physical Chemistry 92:829-833, 1988, and Luisi. P. L. et al Colloid and Polymer Science 268:356-374. 1990, both of which are incorporated herein by reference in their entirety.

The lecithin organogel preferably comprises 1:1 to 1.5:5 (weight/volume) of Phosphlipon 90 (PC) (American Lecithin, Oxford, Conn.) isopropyl palmitate(1 g: 1 ml). water is added to form the desired gel. Other penetrating agents maybe used in the composition of the present invention.

The composition according to the present invention can be in the form of lotions, salves, creams, ointments, liposomes, giant micelles, iontophoresis and salves.

A gelling agent optionally may be added to the formulation. Gelling agents that are suitable include, but are not limited to Pluronic lecithin organogel, cellulose ethers, alginates, polyacrylates, carboxvinyl polymer, bentonite, gelatin, tragcanth, polyvinylpyrrolidone, polyvinyl alcohol, and polyoxyethylene/polyoxtpropylene block copolymers. In this context polyacrylates are used as thickening agents.

The term Pluronic refers to polxamer compound and are sold collectively under the Trade mark PLURONIC, BASF Parsippany, N.J.) PLURONIC F-127 (PL 127) corresponds to poloxamer block copolymer described by Schmolka in the Journal of Biomedical Materials Research 6:571-582, 1972. Other Pluronics maybe used in the present invention. As used in this application, the terms PLURONIC organogel, poloxamer organogel, and polyoxyethylene/polyoxypropylene orgnogel are synomous.

The term “analogue” refers to a chemical compound that is structurally similar to another, but differs slightly in composition (as in the replacement of a different element or in the presence of a particular functional group).

The term “metabolite” refers to a product of metabolism”>

The term “derivative”refers to a chemical substance related structurally to another substance and theoretically: a substance that can be made from another substance.

The term & abbreviation PLURONIC lecithin organogel; PLO refers to the creation of a giant reverse micelle from lower purity phosphatidylcholine than originally taught by Willimann et al. with the use of poloxamer 407. The PLO serves as a transdermal delivery vehicle.

The term “pharmacologically active agent” relates to any chemical material or compound suitable for topical administration which includes any desired local effect on animal or human tissue contacted therewith.

By the term “therapeutically effective amount” of a molecule, drug, or pharmacologically active agent is meant a nontoxic but sufficient amount to provide the desired therapeutic effect.

The “enhanced penetration” caused by the compositions of this invention as in topical application with this method, means increased penetration into the skin, and is achieved with compounds such as lecithin organogel, poloxamer lecithin organogel., or poloxamer phospholipid gel. These include PC, phosphatidylethanolamine, phosphatidylserine, phosphatidylinositol and phosphatidic acid.

“Topical” application is used to mean local administration of the composition and its various embodiments, for example in the treatment of scar. The composition according to the present invention can be in the form of solutions, lotions, salves, creams, ointments, liposomes, giant reverse micelles, sprays, gels, roller sticks, or any other method using giant reverse micelles or micelles and pharmacologically acceptable penetration enhancers.

In one embodiment, the composition may be applied to the scar at bedtime and again before showering in the morning for two applications per day. The composition requires 30 minutes contact time with the scar.

The present invention optionally includes lecithin organogel in combinations with an approximately 15%-40% solutions of Pluronic F-127 (BASF, Parsippany, N.J.), other wise known as poloxamer 407, in a ratio of approximately 17.5% of a 100 g formulation.

Solvents used in the preparation of a variety of gels. Including lecithin gels, all of which are appropriate in practicing the invention, are described in Scartazzini, et al. Journal of Physical Chemistry 92:829-833, and Luisi. P. L., et. al. Colloid and Polymer Science 268: 356-374. 1990, both of which are incorporated here in by reference in their entirety and may be referred to. 1 nM disodium edetate I maybe added to the gel so as to minimize auto oxidation by chelating any heavy metals. However, 50 mg of tetrahydrocurcumin which is a chelator and an excellent anti-oxidant in that it is functional in the aqueous and lipid fraction and is preferred. It is added to the PHOSPHAL 50 after dissolution in a small amount of ethanol.

An improvement in the invention uses a second water phase with a 1 to 2% carbomer and other polyacrylate polymers which act as a cage containing the emollients, humectants and cushion esters to obviate the tackiness of the PLURONIC 127. Ingredients in the carbomer phase are partitioned from the PlURONIC lecithin gel and this prevents collapse of the PLO.

These polymers consist of a water-swellable, high molecular weight, crosslinked homopolymers and copolymers based on acrylic acid, which form hydrogels in aqueous solution. Modifying the crosslink types and levels, as well as amounts and characteristics of the hydrophobic comonomers other polyacrylate polymers are available for the 2^{nd} water phase. These include the traditional carbomer polymers, as well as Noveon polycarbophils and Pemulen polymeric emulsifers all of which may be used in the 2^{nd} water phase.

A 38.5% PLURONIC 127 solution is used and only 45.45 ml of Pl 127 38.5% would be necessary to provide the necessary 17.5 g of PLURONIC per 100 g of formulation. Levels below 17.5 g will not produce a stable gel which is necessary for the transdermal delivey. The use of PLURONIC solutions of a higher concentration allow the use of more actives. A 38.5% PLURONIC allow the use of 54.55 g of actives. This in turn allows the use of 55.45 g of actives which is an increase of 3× the actives contained in a traditional 100 g PLO. This will allow the use of 100 of PLO with 200 g of cabomer gel. This creates an aesthetically acceptable formulation which retains its efficacy.

Examples of Formulations

Pluronic Lecithin Organogel with 8% Phytosphingosine

1. 8. g of phytosphingosine and 50 mg of tetrahydrocurcumin are added to 30 ml of Phosal 50 and magnetic stirring is done at 65° C. until dissolution of the of the phytosphingosine occurs. 10 ml of distilled water is slowly added so as to drive the phytosphingosine between the tails of the phosphatidylcholine by hydrophobic forces. Several minutes of a Braun type mixer or a homogenizer are used. A conventional mix master has also been used with success, but the Braun mixer and the homogenizer are preferred for reducing particulate matter to the smallest possible size. This promotes better bonding and a more effective formulation.

Upon cooling 45.45 ml of PL 127 38.5% solution is added to the above beaker and 0.1 g propylparaben, 0.25 g methylparaben and 0.25 ml DMDM Hydantoin. Power stirring is used to Insure proper distribution of the Pluronic lecithin organogel. 5 minutes would be considered a minimum time. This provides 100 g of formulation.

Preparation of Carbomer Phase

3 to 4 grams of carbomer 940, although other carbomers may be used in the invention are added to 197 ml distilled water with magnetic stirring creating a strong vortex. The carbomer is slowly added to the vortex so that it is all thoroughly whetted. Approximately 1.85 g of Tromethamine is used to adjust the pH to 5.5 and produce the gel.

Ingredients

1. 15 g glyceryl monostearate

2. 8 ml oleyl alcohol

3. 10 ml octyl stearate

4. 15 ml CRODOMOL OS, octyl stearate

5. 15 ml NGCP2 neopenty glycol dipryllate dicaprate

6. 10 g cetyl ester wax

7. 15 ml tridecyl stearate

8. 10 ml ABIL B 8832 polyether-modified polysiloxane

9. 5 g cetyl alcohol

The above ingredients are heated to 70° C. with periodic stirring until the solid lipids are dissolved and a creamy consistency is obtained. The temp is allowed to drop to 35° C. and the preservatives are then added. 0.3 g propylparaben, 0.75 g methylparaben and 0.75 ml DMDM hydantoin. The pH is verified to be 5.5 and adjusted carefully if necessary. 100 g of the 8% Phytosphingosine PLURONIC lecithin organogel is added to the 300 g of the 1% carbomer gel and power stirring is used for a minimum of 5 minutes. 100 g of the mixture contains 2% phytosphingosine with 6 ml of PHOSAL 50 which is very similar to the original work done with only a PLO. Essentially only 25 g. of PLO are used and the tackiness of PLURONIC is not evident.

Compliance is a major issue in medicine and the use of the 2^{nd} water phase allows a patient to comply as directed. In addition, young children may be treated without concern for excoriation of the scars.

A second improvement is the cessation of pruritus which is common in scar because of a dysfunctional barrier lipids. This was reported in by Crandall in 6,306,383. It was assumed that the barrier lipids were restored by topical delivery of the ceramide phytosphingosine. It is now thought that the “enhanced penetration” of the phosphatidylcholine stimulates the cells in the upper epidermis to produce pharmacologically ceramide 1 which restores the barrier. This is very important in that scars have a hypertrophic stratum corneum and the barrier is dysfunctional. This up regulates the proinflammatory cytokines IL-1 & tumor necrosis factor alpha. Once the barrier is restored, the cytokines are down regulated and pruritus ceases.

Tetrahydrocurcumin PLO

21 g of tetrahydrocurcumin is added to a beaker with 10 ml of ethanol and 20.0 ml of PHOSAL 50 and 13. g of PHOSAL 75 heated to 70° C. with magnetic stirring. Power stirring with a mix master or preferably a Braun type mixer or a homogenizer is done to insure adequate distribution of the tetrahydrocurcumin Heating at 70° C. is continued to drive off the ethanol and the beaker is weighed to verify this.

45.5 ml of PL127 38.5% are added upon cooling and mixed with power equipment. 0.1 g. propylparaben, 0.25 g methylparaben and 0.25 ml of DMDM Hydantoin are added. The tetrahydrocurcumin′ will serve as an antioxidant as well as a chelating agent. This is a 100 g formulation of 21% tetrahydrocurcumin PLURONIC lecithin organogel. This will be added to 200 g of a 2.0% carbomer gel.

2 g. of carbomer is added to 150 ml distilled water with a strong vortex created by magnetic stirring. The carbomer is slowly added to the vortex to insure uniform wetting. Once good dispersion is evident. The gel is created by neutralizing the contents with 1.5 g of tromethamine. Slow agitation is used to avoid entrapping air bubbles. It is best to wait for the formulation to reach equilibrium to determine pH measurement and the need for further pH adjustment.

The beaker is then heated to 60° C. and the following ingredients are added:

10 g. glycerly monostearate

4.5 g cetyl alcohol

10 ml oleyl alcohol

10 ml octyl stearate

7.5 ml LIPONATE NPGC-2, neopentyl glycol dipyrlate/dicaprate

5 g cetyl ester wax

Slow power stirring is used to distribute the dissolving solid lipids and once their dissolution is evident the temperature is allowed to cool to 35° C. and 0.2 g propylparaben 0.4 methylparaben and 0.5 ml DMDMhydantoin are thoroughly stirred so as to be evenly distributed.

Clinical evaluation of the transdermal delivery capacity of a PLO with a carbomer gel have been excellent, often with the subjects being very complimentary of the aesthetics of the formulation. A thin film is left on the skin which may be washed off in 30 minutes due to the efficacy of the PlO delivery vehicle. Compliance will be improved, particularly for working people who were reluctant to stain their clothes during the work day and risk embarrassment.

### EXAMPLE 1

A women with extensive stretch marks covering the entire abdomen began treatment with a 5% tetrahydrocurcumin in a standard PLURONIC lecithin organogel. She stated” the original gel, referring to the PLO was uncomfortable and felt sticky for a period of time after application. I was so pleased that the stretch marks were disappearing that I tolerated it.

In early March she was asked to try the PLO with the carbomer gel. She stated that she was pleased with the response of the stretch marks to the gel and that the improvement in the feel of the gel would be well received by other women.

Other clinical examples will be submitted by Declaration.

What is claimed is:

1. A method for treating scar comprising the step of topically applying an effective amount of a Protein Kinase C inhibitor, wherein said Protein Kinase C inhibitor is selected from the group consisting of phytosphingosine-1-phosphate, phytosphingosine hydrochloride, phytosphingosine, lysosphingoids, sphingosine, sphingosine-1 phosphate, sphinganine and sphinganine-1, curcuminoids, tetrahydrocurcumin, apigenenin, and W-7, a pharmaceutically effective penetrating agent selected from the group consisting of lecithin organogel and polaxamer 407 lecithin organogel and a pharmaceutically effective polyacrylate polymer.

2. The method according to claim 1, wherein said scar is selected from the group consisting of hypertrophic and keloid. scars.

3. The method according to claim 1, where in said polyacrylate polymer is selected from a group consisting of carbomer polymer, Noveon polycarbophils polymer and Pemulen polymeric emulsifier.

4. The method according to claim 1, wherein said pharmaceutically effective penetrating agent is in a form selected from the group consisting of gels, creams, sprays, salves, balms, liposomes, giant reverse micelles, pads and iontophoresis.

5. The method according to calm 1, wherein the protein kinase c inhibitor is phytosphingosine and its analogues, derivatives and metabolites.

6. The method according to claim 1, wherein the protein kinase c inhibitor is phytosphingosine -1-phosphate, and its analogues, derivatives and metabolites.

7. The method of claim 1, wherein the protein kinase c inhibitor is phytosphingosine-1-phosphate and its analogues, derivatives and metabolites.

8. The method of claim 1. wherein the protein kinase c inhibitor is sphinganine and its analogues, derivatives and metabolites.

9. The method of claim 1, wherein the protein kinase c inhibitor is sphinganine-1, and its analogues and metabolites.

10. The method of claim 1, wherein the protein kinase c inhibitor are lysosphingoids and their analogues, derivatives and metabolites

11. The method of claim 1, wherein the protein kinase c inhibitor is sphingosine and its analogues, derivatives and metabolites.

12. The method of claim 1, wherein the protein kinase inhibitor sphingosine-1-phosphate and its analogues, derivatives and metabolites.

13. The method of claim 1, wherein the protein kinase c inhibitor is curcuminoids, metabolites and analogues, derivatives and metabolites.

14. The method of claim 1, wherein the protein kinase c inhibitor is tetrahydrocurcumin and analogues derivatives. and metabolites.

15. The method of claim 1, wherein the protein kinase c is apigenin.and its analogues, derivatives and metabolites.

17. The method of claim 1, wherein the protein kinase c inhibitor is a W-7 and analogues, derivatives, and metabolites.
There fore it is submitted that patentable subject matter is clearly present. If the examiner agrees but does not feel that the present claims are technically adequate, applicant respectfully requests that the examiner write acceptable claims pursuant to MPEP 707.o7(j).
